(19) United States
(12) Patent Application Publication
(10) Pub. No.: US 2026/0328747 A1
(43) Pub. Date: Oct. 22, 2026

(54) SYSTEM AND METHOD FOR TRACKING PHYSICAL ATTRIBUTES OF COLLECTED WORK PRODUCTS

(71) Applicant: JBT Marel Corporation, Chicago, IL (US)

(72) Inventors: Cezary J. Mroz, Lewis Center, OH (US); Cameron L. Logan, Columbus, OH (US); Elton Brasil Da Costa, Columbus, OH (US); Wyatt W. Wills, Newark, OH (US)

(73) Assignee: JBT Marel Corporation, Chicago, IL (US)

(21) Appl. No.: 19/648,788

(22) Filed: Apr. 15, 2026

Related U.S. Application Data
(60) Provisional application No. 63/791,105, filed on Apr. 18, 2025.

(51) Int. Cl. G01N 33/02; G06K 7/10

(52) U.S. Cl. CPC G01N 33/02; G06K 7/10297

(57) ABSTRACT

A collected product(s) management system may include a grader configured to associate a collected product(s) with collected product(s) physical attribute information from an incoming supply of products. The system may include a collected product(s) physical attribute information system configured to write collected product(s) physical attribute information associated with a collected product(s) from the incoming supply of products to a data storage device and obtain the collected product(s) physical attribute information from the data storage device. The system may include a product containment machine configured to contain each collected product(s) associated with collected product(s) physical attribute information and identify each contained collected product(s) with corresponding collected product(s) physical attribute information obtained from the corresponding data storage device. The system may include a product movement assembly configured to transport each collected product(s) from the grader to the product containment machine while associating each collected product(s) with the corresponding data storage device.

### CROSS-REFERENCE TO RELATED APPLICATION

This application claims the benefit of U.S. Provisional Application No. 63/791105, filed Apr. 18, 2025, the entire contents of which are incorporated herein by reference.

### BACKGROUND

Packaging machinery is used in many packaging processes, including packaging of food stuff. Equipment for automatically packaging articles such as food stuff generally includes sequential stations at which various sorting, weighing, filling, packaging, and sealing/wrapping/closing operations are used. One or more packaging machines may be provided along the line to perform a required function.

A packaging production line for food stuffs may include a grader to weigh and sort items into a designated location (e.g., bins) based on their weight, type, count, etc. Grader weigh bins can be used for a variety of products, including poultry, fish, fruit, and more. In some examples, items move along a conveyor and are directed into a bin or the like with a paddle. In some examples, items move along a conveyor and are placed on rotating trays, the trays measure the weight of each item, and then the trays rotate to direct the items into the appropriate bins or processing lines. In some examples, items move along a conveyor and are directed into a bin or the like with a robotic arm or other harvester/sorter.

A packaging production line for food stuffs may also include one or more controlled conveyors that advance and/or separate aggregated supplies of food stuffs and packages along the line. For instance, a flighted conveyor may be used to supply batched volumes of food stuffs to a receiving machine, such as a hopper, product distribution system, filling system, etc.

A packaging production line for food stuffs may also include a vertical form fill sealing (VFFS) machine is a type of automated assembly-line product packaging system, commonly used in the packaging industry for food and many other products. The machine can construct plastic bags and stand-up pouches out of a flat roll of film, fill them with product, and seal them. Both solids and liquids can be bagged.

To start the bagging process with a VFFS machine, a horizontal sealing bar creates a “bottom seal” by clamping across a bottom edge of a tube, bonding film together, and cutting off any film below. The product may be either pre-measured by a multi-head weighing system or the sealed tube end may be lowered onto a precision weighing table and the product to be bagged is dispensed through the long conical tube in the center of the bag. When the gross weight of the product-filled bag is reached, filling stops, and the horizontal sealing bar seals the top of the bag, simultaneously forming the bottom of the next bag above. This bag is then cut off from the tube and is now a sealed package, ready to advance onward into the product boxing and shipping processes.

A large, funnel-shaped container or hopper may be used to hold bulk materials before they are dispensed into the bags or pouches, allowing for controlled and consistent filling of the bags by gradually releasing the material through a discharge opening at the bottom.

A packaging production line for food stuffs may also include a tray sealer. A tray sealer is a machine that seals plastic film over trays of food to keep it fresh and prevent contamination. Industrial tray sealers are well known. A typical industrial tray sealer includes an infeed conveyor that advances containers or trays containing product towards a sealing station. An accumulation conveyor receives the containers from the infeed conveyor and operates to space the containers in a predetermined manner. Opposed transfer arms each having spaced apart fingers defining bays close around multiple containers, so that one container is received into each bay, and transfer them from the accumulation conveyor into the sealing station so that they are positioned correctly relative to sealing tools in the sealing station. The arms then move apart allowing the sealing tools to close and seal the trays with film. Following sealing the transfer arms close around the now sealed containers and move them out of the sealing station onto an outfeed conveyor which transports the containers away from the tray sealer.

The trays may be filled with product by a suitable product distribution or filling system, such as a robot or other actuator having a vacuum head or other type of end effect for loading large items such as beef steaks or fish fillets into trays. Other types of products, such as candies, berries, nuts, leafy salad greens, trays may be filled using an overhead hopper, bin or feeder.

Improved systems and methods for managing the supply and packaging of food stuffs and the like are described herein.

### SUMMARY

In some aspects, the techniques described herein relate to a collected product(s) management system for managing containment of a collected product(s), including: a grader configured to associate a collected product(s) with collected product(s) physical attribute information from an incoming supply of products; a collected product(s) physical attribute information system configured to write collected product(s) physical attribute information associated with a collected product(s) from the incoming supply of products to a data storage device and obtain the collected product(s) physical attribute information from the data storage device; a product containment machine configured to contain each collected product(s) associated with collected product(s) physical attribute information and identify each contained collected product(s) with corresponding collected product(s) physical attribute information obtained from the corresponding data storage device; and a product movement assembly configured to transport each collected product(s) from the grader to the product containment machine while associating each collected product(s) with the corresponding data storage device.

In some aspects, the techniques described herein relate to a collected product(s) management system for managing containment of collected product(s), including: a grader configured to associate a collected product(s) having predetermined collected product(s) physical attribute information with a collected product(s) transport component; a collected product(s) physical attribute information system configured to write collected product(s) physical attribute information regarding a collected product(s) to a data storage device and obtain the collected product(s) physical attribute information from the data storage device, each data storage device associated with a collected product(s) transport component; and a product containment machine configured to contain collected product(s) associated with a collected product(s) transport component and identify the contained collected product(s) with the collected product(s) physical attribute information obtained from the data storage device of the associated collected product(s) transport component.

In some aspects, the techniques described herein relate to a food product containment management system for managing containment of collected food product(s) for a containment machine, including: a grader configured to associate a collected product(s) having predetermined collected product(s) physical attribute information with a collected product(s) transport component; a collected product(s) physical attribute information system configured to write collected product(s) physical attribute information regarding a collected product(s) to a data storage device and obtain the collected product(s) physical attribute information from the data storage device, each data storage device associated with a collected product(s) transport component; and a containment machine configured to package collected product(s) associated with a collected product(s) transport component and identify the contained collected product(s) with the collected product(s) physical attribute information obtained from the data storage device of the associated collected product(s) transport component.

In some aspects, the techniques described herein relate to a collected product(s) management system for managing containment of collected product(s), including: a grader configured to associate a collected product(s) having predetermined collected product(s) physical attribute information with a collected product(s) transport component; a collected product(s) physical attribute information system configured to write collected product(s) physical attribute information regarding a collected product(s) to a data storage device and obtain the collected product(s) physical attribute information from the data storage device, each data storage device associated with a collected product(s) transport component; a product containment machine configured to contain collected product(s) associated with a collected product(s) transport component and identify the contained collected product(s) with the collected product(s) physical attribute information obtained from the data storage device of the associated collected product(s) transport component; a processor; and a memory storing instructions that, when executed by the processor, cause a computing device of the collected product management system to: process collected product(s) physical attribute information; and output a collected product(s) containment optimization plan including at least one of recommendations and instructions for adjusting at least one of the containment machine and the grader based on at least one of collected product(s) physical attribute information, containment machine settings, containment machine material data, grader settings, incoming product supply data, and product demand data.

In some aspects, the techniques described herein relate to a method for managing containment of collected product(s), including: associating, with a collected product(s) physical attribute information system, a collected product(s) having predetermined collected product(s) physical attribute information with a collected product(s) transport component; writing, with the collected product(s) physical attribute information, collected product(s) physical attribute information regarding the collected product(s) to a data storage device associated with the collected product(s) transport component; moving the collected product(s) and the collected product(s) transport component to a product containment machine; obtaining, with the collected product(s) physical attribute information system the collected product(s) physical attribute information from the data storage device; containing, with the product containment machine, the collected product(s); and associating, with an identification device, collected product(s) physical attribute information with a container of the collected product(s).

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a block diagram of a non-limiting example of a collected product physical attribute management system.

FIG. 2 is a block diagram of a non-limiting example of a tracking system for a collected product physical attribute management system.

FIG. 3 is a schematic diagram of an exemplary packaging production line for a collected product physical attribute management system.

FIG. 4 is an isometric view of an exemplary containment production line for a collected product physical attribute management system.

FIG. 5 is an isometric view of a portion of the exemplary containment production line of FIG. 4.

FIG. 6 is a block diagram of a non-limiting example of a collected product optimization computing device for a collected product physical attribute management system.

FIG. 7 is a flowchart of a non-limiting example of a method of managing containment of collected product(s) for a containment production line.

FIG. 8 is a block diagram that illustrates a non-limiting example embodiment of a computing device appropriate for use as a computing device with embodiments of the present disclosure.

### DETAILED DESCRIPTION

As discussed above, equipment for automatically packaging articles such as food stuff generally includes sequential stations at which various sorting, weighing, filling, packaging/containment, and sealing/wrapping/closing operations are used. For instance, a grader may be used to weigh and sort item(s) or product(s) into a designated location (e.g., bins) based on their weight, count, etc. The weighed/sorted product(s) may be moved to a receiving machine, such as a hopper, product distribution system, filling system, etc., and/or a containment machine, such as a VFFS machine, a tray sealer, etc. For instance, a flighted conveyor may be used to supply batched volumes of food stuffs to a receiving and/or a containment machine.

A weight of a batch of product(s) may be obtained by a suitable weight measurement device, such as a weigh grader, a scale, a vision system, etc. Similarly, a tallied amount and/or volume of product(s) may be obtained by a suitable sensor device, such as a vision system. Any sensor devices may be coupled to a controller of the corresponding machine.

Typically, receiving and/or packaging/containment machines of a packaging/containment line are controlled by a programmable logic controller (PLC). Data pertaining to the weighed/sorted product(s) may be transmitted from the PLC of the sorting/weighing machine to the PLC of the receiving and/or a packaging/containment machine for use in packaging/containment line management. For instance, a weight, tallied amount and/or volume of a batch of product(s) may be used to determine the type of packaging/containment, the intended use of the items, etc.

Data transfer between machine PLCs is unreliable as the PLCs can get out of sync and data can be easily lost (e.g., during power failures). Moreover, a receiving machine can miss bags, skip bags, miss collection buckets, skip collection buckets, get out of sync, etc. As a result, the PLC data from the weigher/sorter no longer corresponds to the product(s) being received/packaged/contained.

Systems and methods disclosed herein relate to packaging/containment line tracking, management, and optimization. Using the systems and methods disclosed herein, data relating to a product(s) to be packaged may be more reliably and efficiently used to manage and optimize product(s) receipt and/or packaging/containment. Other benefits of the systems and methods disclosed herein will also become appreciated from the descriptions and illustrations provided herein.

Aspects of the disclosed systems and methods will be described with reference to a grader that supplies weighed/sorted product(s) to a receiving machine or portion of a packaging/containment machine. Systems and methods will be specifically described with reference to a VFFS packaging machine and/or a receiving portion of such a machine. However, it should be appreciated that the disclosed systems and methods may be used with any suitable grader and any receiving and/or packaging/containment machine. Further, the disclosed systems and methods may be used with other machines related to item processing, such as a tray sealer, a vacuum sealing machine, etc.

Moreover, the disclosed systems and methods, though sometimes described with specific applicability to food stuffs, may also be used outside of the food processing area. Accordingly, it is to be understood that references to “food stuffs”, “food products”, “products”, “food container”, “bag”, “tray”, etc., should not be seen as limiting.

As noted above, the disclosed systems and methods may be used with a grader that supplies weighed/sorted item(s) to a VFFS packaging machine and/or a receiving portion of such a machine. Any suitable grader generally configured to carry out sorting, weighing, counting, etc., of the product(s), such as food products, may be used. Further, any suitable VFFS containment machine and/or a receiving portion of such a machine generally configured to carry out filling, packaging/containment, sealing/wrapping/closing, etc., may be used. In some examples, the VFFS containment machine is a VFFS machine available from JBT Marel Corporation of Chicago, IL.

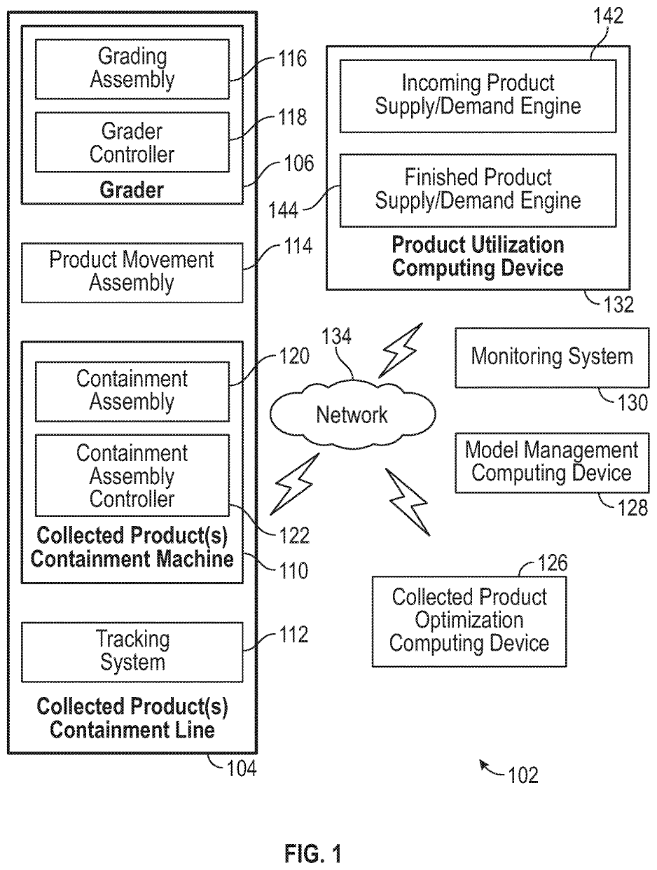

FIG. 1 depicts a block diagram of a non-limiting example of a collected product physical attribute management system 102, or simply, “collected product(s) management system 102”. The collected product(s) management system 102 may include various components and networked computing devices configured for managing aspects of collected products, such as one or more of grading, sorting, collecting, transferring, and containment of products. In the depicted example, the collected product(s) management system 102 includes a collected product(s) containment line 104, a product utilization computing device 132, a monitoring system 130, a model management computing device 128, and a collected product optimization computing device 126 communicatively coupled together through a network 134. The network 134 can be any kind of network capable of enabling communication between the various components of the collected product(s) management system 102. For example, the network can be a WiFi network.

A general overview of the components of the collected product(s) management system 1022 will first be provided. As noted above, the collected product(s) management system 102 is generally configured to carry out and manage aspects of collected product(s), including tracking and managing one or more physical attributes of a collected product(s), such as its size, type, amount, volume, etc.

The collected product(s) containment line 104 used with the collected product(s) management system 102 may be generally configured to carry out grading, transportation of, and at least one of batching and containment of a collected product(s). The collected product(s) containment line 104 may include suitable grading, product movement, and product containment (e.g., batching/containment) assemblies as will be described further herein. The collected product optimization computing device 126 may be generally configured to manage aspects of collected product(s), including grading, movement, and/or containment. The model management computing device 128 may be generally configured to train one or more machine learning models for use in the collected product(s) management system 102. The monitoring system 130 may be generally configured to curate and provide information regarding the collected product(s) management system 102. The product utilization computing device 132 may be generally configured to curate and provide information regarding incoming aggregated product supply and finished product demand to one or more other components, such as the collected product(s) containment line 104, the collected product optimization computing device 126, the model management computing device 128, and/or the monitoring system 130.

It should be appreciated that any of the techniques described herein may be carried out by any suitable computing device(s) and should not be limited to the specific configurations provided herein. For instance, some or all the techniques described herein may be carried out by the product utilization computing device 132 or another computing device. Thus, the examples and techniques discussed herein should not be seen as limiting.

As noted above, the collected product(s) containment line 104 may include suitable grading, product movement, and product containment (e.g., batching/containment) assemblies. In the depicted exemplary block diagram of FIG. 1, the collected product(s) containment line 104 includes a grader 106, a product movement assembly 114, and a collected product(s) containment machine 110.

The grader 106 may be generally configured to grade a product(s), such as by type, weight, size, volume, quantity, etc. In that regard, the grader 106 may include a grading assembly 116 configured to gather physical attribute data of the product(s), such as the type, weight, size, volume, quantity, etc. In that regard, the grading assembly 116 may include a sensor assembly having one or more sensors or measurement devices, such as a scale, image sensors, etc. The grading assembly 116 may obtain and send grading data regarding a product(s) to a grader controller 118, which may process the grading data and, in response to that data, control components of the grader 106.

The grader controller 118 controls grader components to define a collected product(s) having a physical attribute(s) or classification type. More specifically, the grader controller 118 may control robot arms, pneumatic devices, paddles, gates, etc., to deposit a desired volume, quantity, mass, etc., of a product(s) of a certain type, size, weight, etc., into a collection location. For instance, as a product(s) passes by a sensor assembly of the grading assembly 116, such as a weight measurement device and/or a vision system with one or more image sensors, the grader controller 118 may, in response to data regarding the weight, size, shape, color, quantity, type, etc. (“physical attribute(s)”) of the product(s), activate a paddle(s) to move product(s) having a certain physical attribute(s) to a designated chute or hopper. In that regard, “collected product(s)” may be considered to be one or more graded products (e.g., product(s) that have been weighed, imaged, etc.) that are sorted to a designated collection location or otherwise segregated based on a physical attribute(s).

The product movement assembly 114 may include one or more conveyor assemblies configured to move product(s) to, within, and away from the grading assembly 116. A portion of the product movement assembly 114 extending between the grader 106 and the collected product(s) containment machine 110 is configured to maintain segregation of the collected product(s) for subsequent containment.

The collected product(s) containment machine 110 may be generally configured to define a containment for collected product(s), such as by enclosing collected product(s) within a container or package (e.g., a bag, a box, a tray, etc.). Although the term “containment”, “package”, or the like is used to describe the systems and methods used to contain a collected product(s), it should be understood that such terms may be broadly interpreted to include any time of containment or packaging, such as a hopper for batching collected product(s), a tray for receiving collected product(s) to be sealed, a bag, a box, a pouch, a carton, etc.

In the example shown, the collected product(s) containment machine 110 may include a containment assembly 120 configured to receive and/or package the collected product(s). The collected product(s) containment machine 110 may be a VFFS containment machine, as noted above, or any other suitable machine (e.g., a tray sealer). The containment assembly 120 may receive instructions from a containment assembly controller 122. For instance, the containment assembly controller 122 may be used to control a collected product(s) containment speed, such as a filling speed of the collected product(s) containment machine 110 and/or a conveyance speed of the graded product movement system 408 based on predetermined criteria or requirements for the containment.

The containment speed, such as a filling speed of the collected product(s) containment machine 110 and/or a conveyance speed of the graded product movement system 408 may be based on workpiece supply data from an incoming product supply/demand engine 142 of the product utilization computing device 132 and using external order filling techniques, such as that described in U.S. Pat. Nos. 7,672,752 and 8,688,267, incorporated herein. For instance, if an incoming supply of the products is below a threshold volume needed to produce desired packaged products, the filling speed of the collected product(s) containment machine 110 and/or a conveyance speed of the graded product movement system 408 may be adjusted to meet target production goals.

The collected product(s) containment line 104 may also include a collected product(s) physical attribute information system configured to capture and track data regarding one or more physical attributes of a collected product(s), such as its size, type, amount, volume, etc., and use such captured data for managing aspects of the collected product(s). The collected product(s) physical attribute information system may incorporate aspects of the grader 106 configured to obtain physical attribute(s) data for collected product(s), such as the sensor assembly of the grading assembly 116. The collected product(s) physical attribute information system also includes a tracking system 112 configured to associate the physical attribute(s) data (e.g., from the grading assembly 116 and/or the grader controller 118) with the collected product(s) as the collected product(s) are moved throughout the collected product(s) containment line 104.

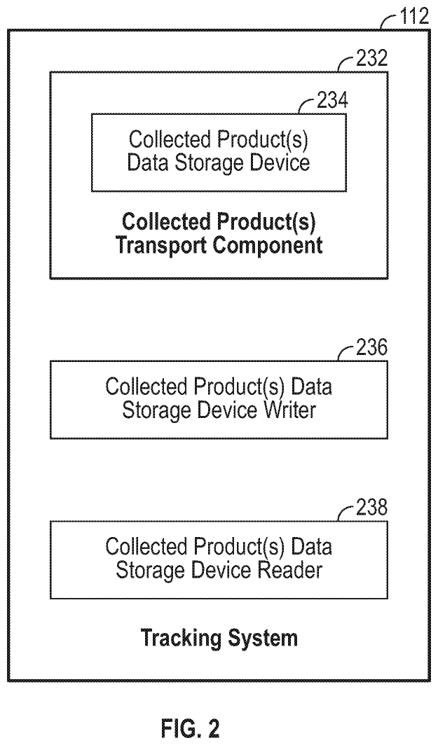

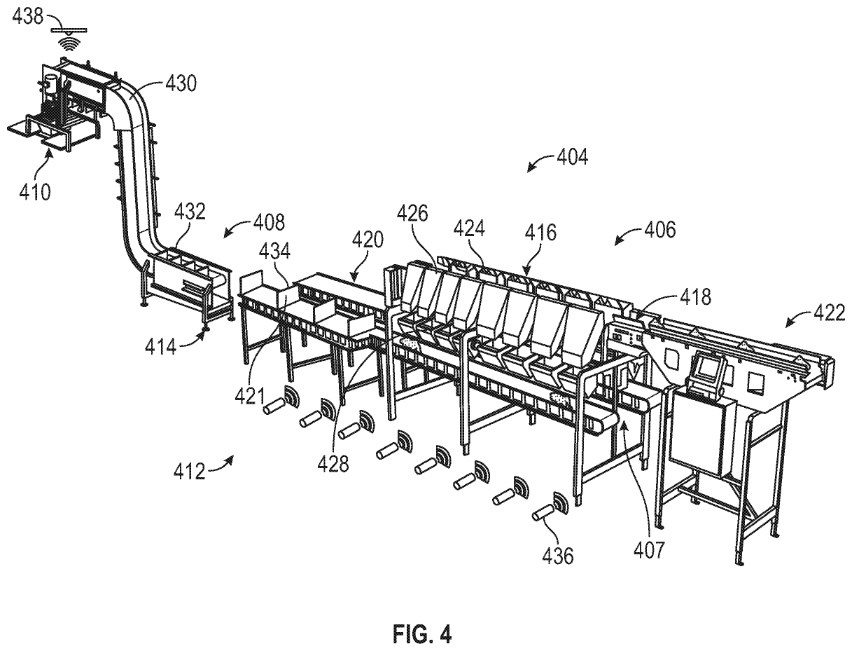

Referring to FIG. 2, the tracking system 112 may include at least one collected product(s) data storage device 234 associated with a corresponding collected product(s) transport component 232. For instance, the collected product(s) data storage device 234 may be a tag (e.g., an RFID tag) or a chip configured to store data associated with a collected product(s). The collected product(s) data storage device 234 may be incorporated into a portion of the product movement assembly 114 configured to move collected product(s). For instance, and as will be illustrated further with the example shown in FIG. 4, an RFID tag or chip may be secured to or integrated into a flight of a flighted conveyor, with each flight configured to move collected product(s). The collected product(s) data storage device 234 may be integrated or embedded within a flight during the conveyor belt molding or forming process.

In some examples, the collected product(s) data storage device 234 may be secured to a flight in a secure manner so as to substantially prevent tampering with the device. In any event, the collected product(s) data storage device 234 may be located on the collected product(s) transport component 232 such that it is within proximity to a reader/writer for reading/writing to the device, such when the device is within a certain proximity of the reader/writer.

The tracking system 112 may further include a collected product(s) data storage device writer 236, such as a tag or chip writer. The collected product(s) data storage device writer 236 may include suitable processing capabilities to write data to a collected product(s) data storage device 234 (e.g., an RFID tag), such as physical attribute information. The collected product(s) data storage device writer 236 may include suitable processing capabilities to first erase any stored information on the collected product(s) data storage device 234 before writing information to the device. In the example of the collected product(s) data storage device 234 being an RFID tag, the collected product(s) data storage device writer 236 may be an RFID tag writer. The output signals of the collected product(s) data storage device writer 236 may be in numeric format, binary format, or another suitable format.

The tracking system 112 may further include a collected product(s) data storage device reader 238, such as a chip reader. The collected product(s) data storage device reader 238 may include suitable processing capabilities to read information stored on a collected product(s) data storage device 234 (e.g., a chip), such as physical attribute information. In the example of the collected product(s) data storage device 234 being an RFID tag, the collected product(s) data storage device reader 238 may be an RFID tag reader.

The collected product(s) data storage device reader 238 may relay such physical attribute information to the containment assembly controller 122 and optionally another computing device, such as the collected product optimization computing device 126, the model management computing device 128, and/or the monitoring system 130.

Referring back to FIG. 1, the physical attribute data for a collected product(s) may be used by the containment assembly controller 122 to control aspects of the containment assembly 120. For instance, the containment assembly controller 122 may direct a printer or labeler to indicate physical attribute data for the packaged, collected product(s) on the sealed package of the collected product(s). In other instances, operational aspects of the containment assembly 120 may be adjusted based on physical attribute information of the collected product(s). For instance, the package closure location may be adjusted based on the overall size or volume of the collected product(s).

The physical attribute data for a collected product(s) may also be sent to another computing device, such as the collected product optimization computing device 126, the model management computing device 128, and/or the monitoring system 130. For instance, the collected product optimization computing device 126 may gather and process physical attribute data for a collected product(s) and use that data to optimize one or more of grading, containment, labeling, and end use of the collected product(s). Further discussion of computer-implemented systems and methods for optimizing one or more of grading, containment, labeling, and end use of the collected product(s) will be described further below with respect to the exemplary collected product optimization computing device 126, the model management computing device 128, and the monitoring system 130.

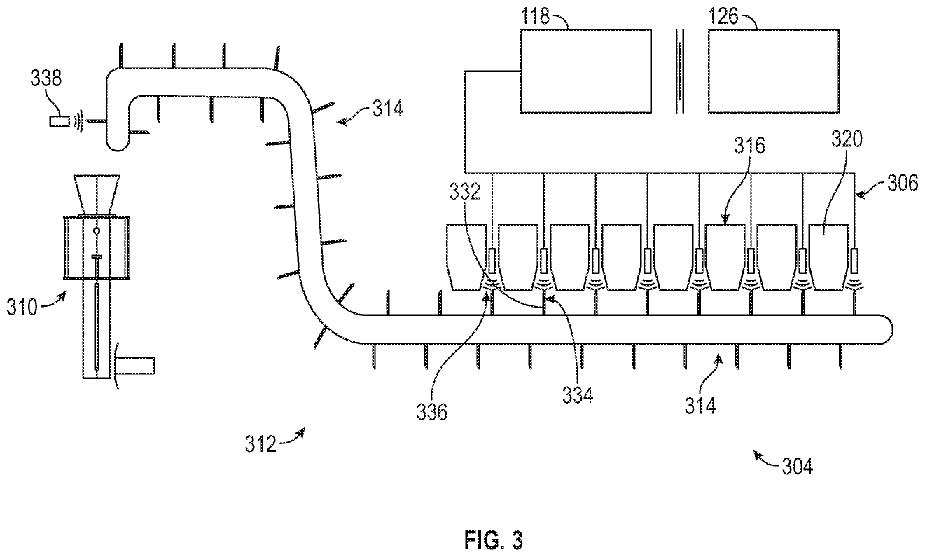

Referring to FIG. 3, a schematic diagram of an exemplary containment production line 304 for food products or similar items will now be described. The exemplary containment production line 304 includes components like that described above with respect to the collected product(s) containment line 104. In that regard, like reference numerals will be used to designate like parts except in the '300 series. Of course, any suitable containment production line may be used with the systems and methods disclosed herein.

The exemplary containment production line 304 includes a grader 306 to grade, weigh, and/or sort products into a designated location (e.g., bins) based on their physical attribute(s), such as weight, type, count, etc. The exemplary containment production line 304 further includes a containment machine 310 for receiving and/or containment the weighed/counted/sorted product(s) (the collected product(s)) received from the grader 306. Finally, in the example shown, the containment production line 304 includes a product movement assembly 314 configured to transport product to, within and away from the grader 306.

In the example shown, the grader 306 includes a grading assembly 316 and a grader conveyance system having an endless conveyor configured to transport product to, within and away from the grading assembly 316. In the depicted example, the conveyance system of the grader 306 is defined by an endless conveyor(s) of the product movement assembly 314.

The product movement assembly 314 may be generally configured to receive and transport an aggregated supply of products toward the grading assembly 316 of the grader 306. In that regard, the product movement assembly 314 may receive an aggregated supply of products from any suitable upstream bulk transfer assembly or loading system, such as a conveyor, a bin tipping system, a tote dumper, an elevated feed conveyor/hopper system, a pump feed system, manual labor, etc.

The product movement assembly 314 may be configured to transport the aggregated supply of products through the grading assembly 316 of the grader 306. As the aggregated products are graded by the grading assembly 316 (such as with a dynamic scale, a vision system, etc.), the products may be sorted to one or more bins, chutes, etc., based on the grade of the product(s). In that regard, the product movement assembly 314 may move the aggregated products generally linearly through the grading assembly 316 at a suitable speed to support the necessary grading.

The product movement assembly 314 is also configured to transport graded or collected product(s) to the containment machine 310. In that regard, the product movement assembly 314 may include vertically protruding flights 332 or other product separation structure to maintain segregation of collected product(s) as they are transported. In some examples, a separate outfeed conveyor is located beneath the grading assembly 316 to receive collected product(s).

It should be appreciated that the term “flight,” as used herein, may include the substantially isolated space defined on the conveyor to maintain segregation of collected product(s) as they are transported, and/or the vertically protruding structure configured to define bounds of the substantially isolated space on the conveyor. Thus, the descriptions and illustrations should not be seen as limiting.

The grading assembly 316 may be any suitable automated, semi-automated, and/or manual assembly that is configured to grade or designate a product(s) use based on one or more measured/sensed product parameters defining physical attribute(s) (e.g., weight, type, size, shape, etc.). The grader may sort products using a pivoting paddle, pneumatics, a robotic arm, etc. In some examples, the grading assembly 316 may be a grading assembly available from JBT Marel Corporation.

The graded, collected product(s) may be directed into a collection location, such as one of a plurality of product chutes, bins, hoppers, etc. In the example shown, the collected product(s) are directed into one of a plurality of bins or hoppers 320, such as after passing through a product chute.

The hopper 320 is configured to receive/collect a predetermined volume of product(s). Upon receiving a predetermined volume of product(s), the hopper 320 can selectively deposit the collected product(s) onto a designated portion of the product movement assembly 314 (such as an outfeed conveyor). For instance, a gate of the hopper 320 may be selectively opened to deposit collected product(s) onto a flight 332 of the product movement assembly 314 when the designated conveyor flight is positioned beneath the hopper 320 (determined, for instance, with an encoder).

The grade or product designation may be defined in response to instructions from a grader controller (e.g., grader controller 118) and/or a computing device(s) in communication with the grader controller and/or the grading assembly 316 (e.g., the collected product optimization computing device 126), as shown. For instance, the grading assembly 316 may activate sorting components to sort products based on one or more measured or sensed physical attribute(s) to meet certain containment fill requirements for the products. The containment fill requirements may be defined, for instance, by a product optimization computer (e.g., the collected product optimization computing device 126) based on various inputs and data.

The collected product(s) are transported via the product movement assembly 314 to the containment machine 310. The product movement assembly 314 delivers collected product(s) to the containment machine 310 for filling and closing. The containment machine 310 may be a VFFS containment machine, as noted above. In that regard, the containment machine 310 may be configured to seal a bag after receiving a collected product(s) from a flight 332 of the product movement assembly 314. The containment machine 310 may include suitable sensors and controls to activate a sealing/closing mechanism after the product movement assembly 314 has completed the deposit of the collected product(s).

The exemplary containment production line 304 further includes a tracking system 312 configured to capture collected product(s) physical attribute information and transfer the information from the grader 306 to at least the containment machine 310. In that regard, the tracking system 312 may include a data reader/writer device 336, such as an RFID tag writer, as discussed above with respect to FIG. 2. The data reader/writer device 336 may be positioned relative to the product movement assembly 314 to write data to a data storage device 334 secured to or embedded within a flight 332 of the product movement assembly 314 (optionally after erasing any data stored thereon). The data written to the data storage device 334 may correlate to physical attribute(s) of the collected product(s).

The tracking system 312 may further include a collected product(s) data storage device writer (and optionally reader), or a data reader/writer device 338, such as an RFID tag reader, as discussed above with respect to FIG. 2. The data reader/writer device 338 may be positioned near an inlet of the containment machine 310. In that manner, data pertaining to the collected product(s) to be delivered into the containment machine 310 can be used by the containment controller (e.g., see containment assembly controller 122) to control, monitor, and/or manage aspects of the containment.

The physical attribute information of the collected product(s), retrieved from the data reader/writer device 338, may be used (e.g., by the containment assembly controller 122) to code, label, or otherwise identify the packaged collected product(s). The data reader/writer device 338, may also relay such physical attribute information to another computing device, such as the collected product optimization computing device 126, the model management computing device 128, and/or the monitoring system 130. However, no transfer of data between the grader 306 and the containment machine 310 is needed to associate physical attribute data of collected product(s) with a packaged collected product(s).

Referring to FIG. 4, an isometric view of an exemplary containment production line 404 for food products or similar items will now be described. The exemplary containment production line 404 includes components like that described above with respect to the collected product(s) containment line 104 and containment production line 304. In that regard, like reference numerals will be used to designate like parts except in the '400 series. Of course, any suitable containment production line may be used with the systems and methods disclosed herein.

The exemplary containment production line 404 includes a grader 406 to grade, weigh, and/or sort products into a designated location (e.g., bins) based on their weight, type, count, etc. The exemplary containment production line 404 further includes a containment machine 410 for receiving and/or containment the weighed/counted/sorted product(s) (the collected product(s)) received from the grader 406. Finally, in the example shown, the containment production line 404 includes a product movement assembly 414 having a grader conveyance system 407 and a graded product movement system 408 configured to transport product to, within and away from the grader 406.

In basic form, the grader 406 includes a grading assembly 416 and the grader conveyance system 407 having a plurality of endless conveyors configured to transport product to, within and away from the grading assembly 416. In the depicted example, the grader conveyance system 407 of the grader 406 includes an infeed conveyor 422, a graded product movement system 408, and an outfeed conveyor 420.

The infeed conveyor 422 may be generally configured to received and transport an aggregated supply of products toward the grading assembly 416 of the grader 406. In that regard, the infeed conveyor 422 may receive an aggregated supply of products from any suitable upstream bulk transfer assembly or loading system, such as a conveyor, a bin tipping system, a tote dumper, an elevated feed conveyor/hopper system, a pump feed system, manual labor, etc.

The graded product movement system 408 may be configured to receive an aggregated supply of products from the infeed conveyor 422 and transport the products through the grading assembly 416 of the grader 406. The graded product movement system 408 may be generally coaxially aligned with the infeed conveyor 422 with minimal gap defined between a downstream end of the infeed conveyor 422 and an upstream end of the graded product movement system 408. The graded product movement system 408 may rotate at the same speed as the infeed conveyor 422, or it may instead rotate faster than the infeed conveyor 422 to help spread out products on the conveyance surface as they are transported to the grading assembly 416. In some examples, the graded product movement system 408 and the infeed conveyor 422 are the same endless conveyor.

The graded product movement system 408 is configured to move products through the grading assembly 416 of the grader 406. As the aggregated products are graded by the grading assembly 416 (such as with a dynamic scale, a vision system, etc.), the products may be sorted to one or more bins, chutes, etc., based on the grade of the product(s). In that regard, the graded product movement system 408 may move the aggregated products generally linearly through the grading assembly 416 at a suitable speed to support the necessary grading.

The outfeed conveyor 420 may be located generally below the grading assembly 416 to transport graded or collected product(s) to the graded product movement system 408. In that regard, the outfeed conveyor 420 may include vertically protruding flights 421 or other product separation structure to maintain segregation of collected product(s) as they are transported. In some examples, the outfeed conveyor 420 is defined by an upstream portion of the graded product movement system 408.

The grading assembly 416 may be any suitable automated, semi-automated, and/or manual assembly that is configured to grade or designate a product(s) use based on one or more measured/sensed product parameters (e.g., weight, type, size, shape, etc.). The grader may sort products using a pivoting paddle, pneumatics, a robotic arm, etc. As noted above, in some examples, the grading assembly 416 may be a grading assembly available from JBT Marel Corporation.

In the depicted example, the grading assembly 416 includes a plurality of pivot paddles 424 located next to the graded product movement system 408 and having a pivot axis generally transverse to the conveyance direction. As a product moves with the conveyance surface of the graded product movement system 408, a pivot paddle 424 sweeps across the conveyance surface to direct the product into one of a plurality of product chutes 426. Each product chute 426 extends between the graded product movement system 408 and a corresponding product receiving hopper 428.

The hopper 428 is configured to receive/collect a predetermined volume of product(s) deposited via the product chute 426. Upon receiving a predetermined volume of product(s), the hopper 428 can selectively deposit the collected product(s) onto a designated portion of the outfeed conveyor 420. For instance, a gate of the hopper 428 may be selectively opened to deposit collected product(s) onto a flight 421 of the outfeed conveyor 420 when the designated conveyor flight is positioned beneath the hopper 428 (determined, for instance, with an encoder).

As can be seen, the grading assembly 416 may include a plurality of pivot paddles 424 and corresponding product chutes 426 and hopper 428 on each side of the graded product movement system 408. In that regard, the outfeed conveyor 420 may include first and second conveyors located on each side of the graded product movement system 408 beneath the corresponding hopper 428.

The grade or product designation may be defined in response to instructions from a grader controller (e.g., grader controller 118) and/or a computing device(s) in communication with the grader controller and/or the grading assembly 416 (e.g., the collected product optimization computing device 126). For instance, the grading assembly 416 may activate sorting components to sort products based on one or more measured or sensed product parameters to meet certain containment fill requirements for the products. The containment fill requirements may be defined, for instance, by a product optimization computer (e.g., the collected product optimization computing device 126) based on various inputs and data.

The collected product(s) are transported via the graded product movement system 408 of the product movement assembly 414 to the containment machine 410. Like the outfeed conveyor 420, the graded product movement system 408 may be a flighted conveyor configured to maintain segregation of collected product(s) as they are transported. In that regard, the graded product movement system 408 may include an endless conveyor belt 430 having vertically protruding flights or other product separation structure to maintain segregation of collected product(s). Although the graded product movement system 408 is shown as a separate conveyor, the graded product movement system 408 may instead simply be a continuation of the outfeed conveyor 420. If separate, the graded product movement system 408 and outfeed conveyor 420 may be timed or otherwise controlled to smoothly transfer collected product(s) from the outfeed conveyor 420 to the graded product movement system 408.

The graded product movement system 408 delivers collected product(s) to the containment machine 410 for filling and closing. The containment machine 410 may be a VFFS containment machine, as noted above. In that regard, the containment machine 410 may be configured to seal a bag after receiving a collected product(s) from a flight of the graded product movement system 408. The containment machine 410 may include suitable sensors and controls to activate a sealing/closing mechanism after the graded product movement system 408 has completed the deposit of the collected product(s).

The exemplary containment production line 404 further includes a tracking system 412 configured to capture collected product(s) physical attribute information and transfer the information from the grader 406 to at least the containment machine 410. In that regard, the tracking system 412 may include a collected product(s) data storage device writer 436, such as an RFID tag writer, as discussed above with respect to FIG. 2. The collected product(s) data storage device writer 436 may be positioned relative to the outfeed conveyor 420 of the grader 406 to write data to a data storage device 434 secured to or embedded within a flight 421 of the outfeed conveyor 420 (optionally after erasing any data stored thereon). The data written to the data storage device 434 may correlate to physical attribute(s) of the collected product(s).

The tracking system 412 may further include a data reader/writer device 438, such as an RFID tag reader, as discussed above with respect to FIG. 2. The data reader/writer device 438 may be positioned near an inlet of the containment machine 410. In that manner, data pertaining to the collected product(s) to be delivered into the containment machine 410 can be used by the containment controller (e.g., see containment assembly controller 122) to control, monitor, and/or manage aspects of the containment.

The data reader/writer device 438 may also or instead be positioned near an upstream end of the graded product movement system 408 if the graded product movement system 408 is separate from the outfeed conveyor 420. In that manner, data pertaining to the collected product(s) may be tied to a particular flight of the graded product movement system 408, which may then be used by the containment assembly controller 122. In such an example, an additional collected product(s) data storage device writer 436 may be used to write data to a data storage device secured to or embedded within a flight of the graded product movement system 408 (optionally after erasing any data stored thereon).

The physical attribute information of the collected product(s), retrieved from the data storage device 434, may be used (e.g., by the containment assembly controller 122) to code, label, or otherwise identify the packaged collected product(s). As noted above, the data reader/writer device 438 may relay such physical attribute information to another computing device, such as the collected product optimization computing device 126, the model management computing device 128, and/or the monitoring system 130. However, no transfer of data between the grader 406 and the containment machine 410 is needed to associate physical attribute data of collected product(s) with a packaged collected product(s).

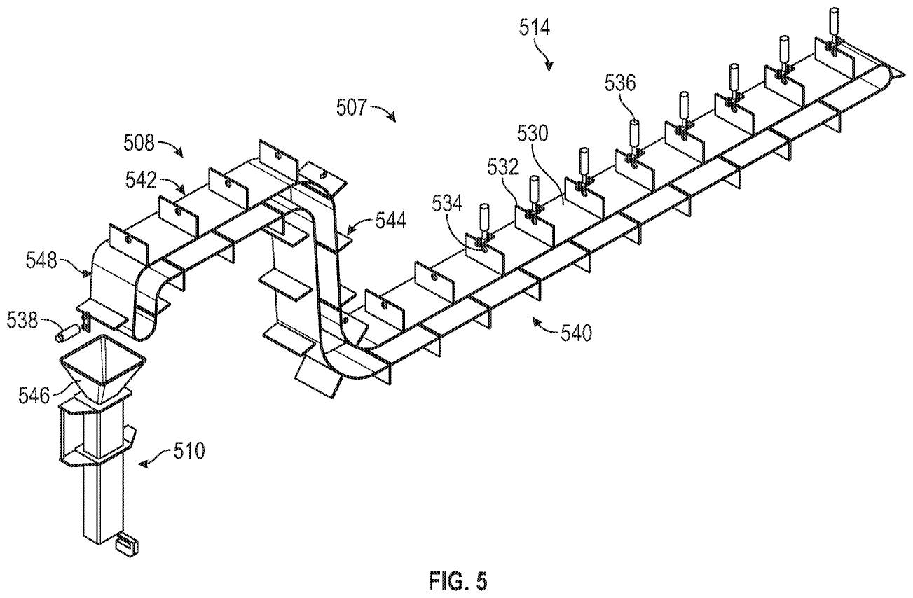

FIG. 5 shows an isometric view of a product movement assembly 514 for use with a grader (not shown) and a containment machine 510, such as the grader and containment machines described herein. The product movement assembly 514 includes a grader conveyance system portion 507 and graded product movement system portion 508 that are integrated together into a continuous movement assembly, such as what may be used for the containment production line 404 shown in FIG. 4.

The exemplary product movement assembly 514 includes components like that described above with respect to the collected product(s) containment line 104, the containment production line 304, and the exemplary containment production line 404. In that regard, like reference numerals will be used to designate like parts except in the '500 series. Of course, the product movement assembly 514 may be used with any suitable containment production line.

Unlike in the product movement assembly 414, the product movement assembly 514 includes a grader conveyance system portion 507 and product movement system portion 508 defined by a single continuous endless conveyor belt. In that regard, the product movement system portion 508 can be seen as the same as or an extension of the grader conveyance system portion 507 (and/or any outfeed conveyor) of the grader. Of course, the grader conveyance system portion 507 and product movement system portion 508 may instead be defined by separate conveyors. Moreover, the grader conveyance system portion 507 may include any infeed conveyors, or it may be independent of infeed conveyors.

As can be seen, the product movement assembly 514 includes an endless conveyor belt 530 with a plurality of flights 532 for separating collected product(s). The product movement assembly 514 includes a first substantially horizontal portion 540 at a first height, a second substantially horizontal portion 542 at a second height higher than the first height, and a substantially vertical or diagonal intermediary portion 544 extending between the first and second substantially horizontal portions. In that regard, collected product(s) can be deposited onto a flighted portion of the first substantially horizontal portion 540 of the grader conveyor 518 and transported to an elevated position by the intermediary portion 544. The second substantially horizontal portion 542 may be positioned to deliver the collected product(s) to an elevated portion of the containment machine 510, such as a containment hopper 546.

A substantially vertical portion 548 may be defined at the downstream end of the second substantially horizontal portion 542. The substantially vertical portion 548 may be positioned relative to the containment hopper 546 to deposit collected product(s) on the product movement assembly 514 substantially vertically downwardly into the containment hopper 546. In that manner, collected product(s) simply falls off the product movement assembly 514 and into the containment hopper 546 as the conveyor wraps around a downstream roller defined at the end of the substantially vertical portion 548. Of course, it should be appreciated that any other suitable product movement assembly may instead be used.

The collected product(s) data storage device writer 536 of the tracking system 512 may be located near the first substantially horizontal portion 540. As shown, the collected product(s) data storage device writer 536 may be embodied as a plurality of device writers, each spaced relative to one another, such as a distance substantially equal to a flighted portion of the product movement assembly 514. In this manner, each collected product(s) data storage device writer 536 can write data to a corresponding data storage device 534 secured to or embedded within a flight 532 of the endless conveyor belt 530 (optionally after erasing any data stored thereon).

The data can be written to each data storage device 534 as the collected product(s) are deposited onto the corresponding flighted portion. For instance, a collected product(s) data storage device writer 536 positioned relative to a first, downstream flight 532/data storage device 534 may write data to the first, downstream data storage device 534 regarding physical attribute information of the collected product(s) between the first, downstream flight 532 and a second, upstream flight. At the same or a different time, a collected product(s) data storage device writer 536 positioned relative to the second, upstream data storage device 534 regarding physical attribute information of the collected product(s) between the second, upstream flight and a third flight further upstream from the second flight. In that regard, each collected product(s) data storage device writer 536 may individually or simultaneously write data to the corresponding data storage device 534 as collected product(s) are deposited onto a corresponding flighted portion of the product movement assembly 514.

The data reader/writer device 538 may be positioned near an inlet of the containment machine 510 to retrieve data pertaining to the collected product(s) to be delivered into the containment machine 510. For instance, the data reader/writer device 538 may be positioned above the containment hopper 546 to obtain data for each collected product(s) that is deposited into the hopper from the product movement assembly 514. In that regard, the containment machine 510 can obtain and use physical attribute data relevant to the deposited collected product(s) for managing containment of that collected product(s).

As also noted above, the product movement assembly 514 includes a substantially vertical portion 548 positioned relative to the containment hopper 546 such that collected product(s) can fall from the product movement assembly 514 into the containment hopper 546 as the endless conveyor 530 turns around the downstream roller. In that regard, the data reader/writer device 538 may be positioned relative to the substantially vertical portion 548 to read the data from the data storage device 534 in each flight 532 as the collected product(s) are deposited into the containment hopper 546.

Data pertaining to the collected product(s) delivered into the containment machine 510 can be used by the containment controller (e.g., see containment assembly controller 122) to control, monitor, and/or manage aspects of the containment. For instance, the physical attribute information of the collected product(s), retrieved from the data storage device 534, may be used to code, label, or otherwise identify the packaged collected product(s). The data reader/writer device 538 may relay such physical attribute information to another computing device, such as the collected product optimization computing device 126, the model management computing device 128, and/or the monitoring system 130. However, no transfer of data between the grader and the containment machine 510 is needed to associate physical attribute data of collected product(s) with a packaged collected product(s).

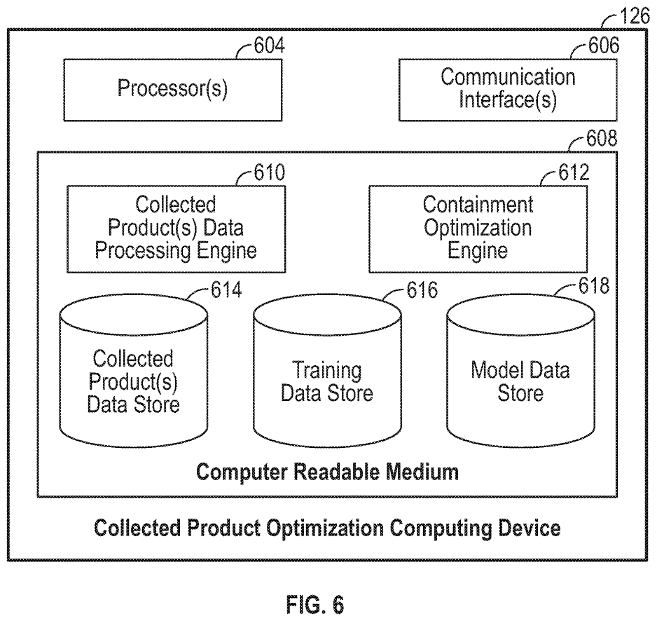

Referring to the block diagram shown in FIG. 6, exemplary aspects of the collected product optimization computing device 126 will now be described. In the exemplary block diagram of FIG. 6, the collected product optimization computing device 126 includes a processor(s) 604, a communication interface(s) 606, computer readable medium 608, and at least one data store (e.g., collected product(s) data store 614, training data store 616, and model data store 618). As shown, the computer readable medium 608 has stored thereon logic that, in response to execution by the one or more processor(s) 604, cause the collected product optimization computing device 126 to provide a collected product(s) data processing engine 610 and a containment optimization engine 612.

Exemplary aspects of the collected product optimization computing device 126 components shown in FIG. 6 will be described with reference to the collected product(s) containment line 104 and tracking system 112 components shown in FIGS. 1 and 2. However, it should be appreciated that any suitable containment production line and tracking system components may be used.

The collected product optimization computing device 126 may be implemented by any computing device or collection of computing devices, including but not limited to a desktop computing device, a laptop computing device, a mobile computing device, an edge computing device, a server computing device, a computing device of a cloud computing system, and/or combinations thereof. In some examples, the processor(s) 604 may include any suitable type of general-purpose computer processor. In some examples, the processor(s) 604 may include one or more special-purpose computer processors or AI accelerators optimized for specific computing tasks, including but not limited to graphical processing units (GPUs), vision processing units (VPTs), and tensor processing units (TPUs).

In some examples, the communication interface(s) 606 includes one or more hardware and/or software interfaces suitable for providing communication links between components. The communication interface(s) 606 may support one or more wired communication technologies (including but not limited to Ethernet, FireWire, and USB), one or more wireless communication technologies (including but not limited to Wi-Fi, WiMAX, Bluetooth, 2G, 3G, 4G, 5G, and LTE), and/or combinations thereof.

As used herein, “computer-readable medium” refers to a removable or nonremovable device that implements any technology capable of storing information in a volatile or non-volatile manner to be read by a processor of a computing device, including but not limited to: a hard drive; a flash memory; a solid state drive; random-access memory (RAM); read-only memory (ROM); a CD-ROM, a DVD, or other disk storage; a magnetic cassette; a magnetic tape; and a magnetic disk storage.

As used herein, “engine” refers to logic embodied in hardware or software instructions, which can be written in one or more programming languages, including but not limited to C, C++, C #, COBOL, JAVA™, PHP, Perl, HTML, CSS, JavaScript, VBScript, ASPX, Go, and Python. An engine may be compiled into executable programs or written in interpreted programming languages. Software engines may be callable from other engines or from themselves. Generally, the engines described herein refer to logical modules that can be merged with other engines or can be divided into sub-engines. The engines can be implemented by logic stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device.

As used herein, “data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

The collected product(s) data processing engine 610 of the collected product optimization computing device 126 may be configured to pre-process incoming data for a collected product(s) and any related data. The data may include size, type, count, volume, mass, etc., of the collected product(s). The related data may include data pertaining to the collected product(s) containment machine 110 (e.g., settings, containment material (e.g., type, availability, etc.), etc.) the grader 106 (e.g., settings), the size, type, and/or location of the collected product(s) transport component 232, etc. Pre-processing of the data may include extracting relevant information, correlating information, compressing data size, scrubbing the data, etc. Such pre-processed data may be stored in the collected product(s) data store 614 for later retrieval.

The data received and pre-processed by the collected product(s) data processing engine 610 may be sent to or retrieved by the containment optimization engine 612, which may use the data to determine a collected product(s) containment optimization plan, such as a collected product(s) containment management recommendations or instructions. The collected product(s) containment management recommendations or instructions may relate to optimizing one or more of grading, containment, labeling, and end use of the collected product(s). The containment optimization engine 612 may output instructions to the containment assembly controller 122 and/or grader controller 118 of the collected product(s) containment machine 110 and/or grader 106, respectively, for automatically or semi-automatically carrying out an action according to the instructions. The containment optimization engine 612 may also or instead output instructions to the containment optimization engine 612 to display a recommendation associated with the collected product(s) containment machine 110 and/or the grader 106 such that an operator may carry out any necessary actions.

In some examples, the collected product(s) containment management recommendations or instructions may include a recommendation to use a certain grading specification for collected product(s), such as based on containment constrictions or requirements (collected product(s) containment machine 110 efficiency, containment material specifications, etc.). If a containment machine is operating with a certain containment material suitable for a grade of lower or higher volume, the recommendation may be to adjust the grading criteria accordingly.

In some examples, the collected product(s) containment management recommendations or instructions may include instructions for sorting packaged, labeled collected product(s) of a first classification to a first end use and packaged, labeled collected product(s) of a second classification to a second end use. The first classification may include collected product(s) of a first size, type, count, volume, etc., and the second classification may include collected product(s) of a second size, type, count, volume, etc. The recommendations or instructions may be based in part on related information retrieved from the incoming product supply/demand engine 142 of the product utilization computing device 132, such as incoming product information (e.g., batch size, type, etc.). The recommendations or instructions may also or instead be based in part on related information retrieved from the finished product supply/demand engine 144 of the product utilization computing device 132, such as fulfillment requirements or orders. The incoming supply and/or demand information from the product utilization computing device 132 can be used by the collected product optimization computing device 126 together with physical attribute data of each packaged, collected product(s) to determine an end use of the package.

As a specific example, if the physical attribute data of the collected product(s) indicates a higher than normal size of chicken breast fillets in the package for certain packages, the finished product supply/demand engine 144 together with the containment optimization engine 612 may designate packages with the larger chicken breast fillets to end uses where the value is based on the chicken breast fillet size. The value may be determined, in part, from information retrieved from the incoming product supply/demand engine 142 regarding the incoming supply of products.

In some examples, the collected product(s) containment management recommendations or instructions may include instructions for adjusting components or settings of the collected product(s) containment machine 110. For instance, if physical attribute data of collected product(s) indicates a larger volume than expected, the timing of the containment machine closure mechanism may be adjusted to increase the size of the package to accommodate such increased volume of collected product(s).

In some examples, if the collected product(s) data storage device reader 238 cannot retrieve or read information from a collected product(s) data storage device 234 for “X” number of consecutive times, indicating collected product(s) were not deposited into the flighted conveyor portion, the containment optimization engine 612 may output instructions to the containment assembly controller 122 of the collected product(s) containment machine 110 to cause one or more components of the collected product(s) containment machine 110 to cease functioning, to function in a different manner, etc. For instance, the containment optimization engine 612 may output instructions to the containment assembly controller 122 to cause the collected product(s) containment machine 110 to stop running the product movement assembly 114 and the containment assembly 120, to run the product movement assembly 114 and the containment assembly 120 at a slower speed or rate, etc., if a threshold absence of collected product(s) data is detected.

As noted above, the physical attribute data for a collected product(s) may also be sent to another computing device, such as the model management computing device 128 and/or the monitoring system 130. For instance, the (optionally pre-processed) physical attribute data for a collected product(s) may be sent to or retrieved by the model management computing device 128 for training one or more containment optimization machine learning model(s) to output containment management recommendations or instructions. The containment optimization machine learning model(s) may be trained to output a collected product(s) containment optimization plan that includes recommendations or instructions to adjust component(s) or setting(s) of the collected product(s) containment machine 110 and/or the grader 106 based on at least one of physical attribute data of collected product(s), containment machine settings, containment material data, grader settings, incoming product supply data, and product demand data. The containment optimization engine 612 may execute the one or more containment optimization machine learning models after retrieving the model(s) from a data store, such as the model data store 618.

The containment optimization machine learning model(s) may be trained by the model management computing device 128, for example, using historical data of initial and corrected settings used for containment machines and/or graders with certain collected product(s) physical attribute data, incoming product supply data, product demand data, etc. Any relevant training data for the containment optimization machine learning model(s) generated by the collected product optimization computing device 126 may be stored in the training data store 616 of the collected product optimization computing device 126 and/or a training data store of the model management computing device 128. After being trained at least initially, the containment optimization machine learning model(s) may be stored on the model management computing device 128 and retrieved by the containment optimization engine 612 for execution, and/or the containment optimization machine learning model(s) may be stored locally on the collected product optimization computing device 126. To support processing of the machine learning models, the collected product optimization computing device 126 may be configured as a local, high power or edge computing device (e.g., like the data processing computing device described in U.S. Provisional Patent No. 63/588,947).

In some examples, the containment optimization machine learning model(s) may be trained using containment line optimization score(s). For instance, containment line optimization score(s) may be correlated to collected product(s) physical attribute data, incoming product specifications (e.g., type, size, etc.), finished package specifications (percentage within spec, yield data, etc.), collected product(s) containment machine 110 initial and adjusted settings, grader 106 initial and adjusted settings, etc. For instance, if, based on a first containment optimization score for a packaged collected product(s) having first specifications, a setting(s) for the collected product(s) containment line 104 is adjusted to generate a different packaged product result, the collected product(s) containment line 104 adjustments can be correlated to the containment line optimization score(s) for a specific collected product(s) containment line 104 configuration.

The collected product(s) containment line 104 setting adjustment data may be derived from operator input in response to a containment line optimization score(s) output and/or a collected product(s) containment optimization plan output from a machine learning model correlated to a score. For example, if an operator receives a collected product(s) containment optimization plan including a recommendation for a collected product(s) containment line 104 machine setting adjustments (optionally with a score), etc., the operator may accept the recommendation, reject the recommendation, and/or make manual adjustments based on the recommendation. The operator's input can be part of the training data.

Any suitable type of artificial intelligence may be used, including machine learning models that incorporate convolutional neural networks and/or computer vision and/or image segmentation, optionally incorporating deep learning techniques. Any suitable technique may be used to train the machine learning models, including but not limited to one or more of gradient descent, data augmentation, hyperparameter tuning, and freezing/unfreezing of model architecture layers. In some examples, annotated, raw images are used as the training input. In some examples, one or more features derived from the images, including but not limited to versions of the images in a transformed color space, set of edges detected in the image, one or more statistical calculations regarding the overall content of the images, or other features derived from the images may be used instead of or in addition to the annotated raw images to train the machine learning models.

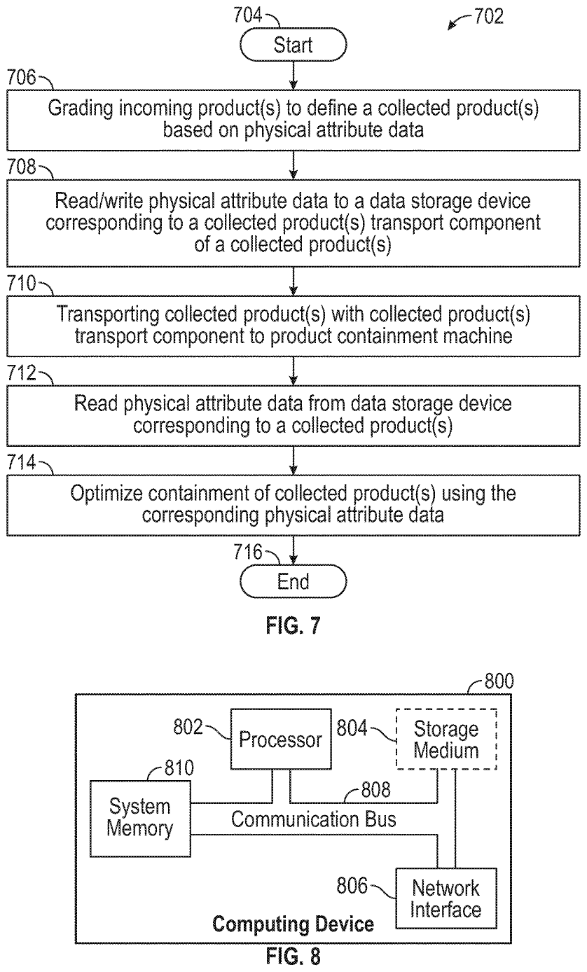

FIG. 7 is a flowchart of a non-limiting example of a method 702 of managing containment of collected product(s) for a containment production line. The method 702 may be carried out using any components described herein with reference to the collected product(s) containment line 104, containment production line 304, containment production line 404, or any other suitable components. Thus, although aspects of the method 702 will be described with reference to the collected product(s) containment line 104 and tracking system 112, the method 702 may be carried out using any of the exemplary implementations of a containment production line and tracking system shown in FIGS. 3, 4, or 5, or a similar containment production line. Moreover, aspects of the method 702 may be carried out by the containment assembly controller 122, the grader controller 118, the collected product optimization computing device 126, the model management computing device 128, and/or any other suitable computing device.

From a start block 704, the method 702 may proceed to block 706, which includes grading incoming product(s) to define one or more collected product(s) based on physical attribute data of the incoming product(s). Grading may be performed by a grading assembly, such as grading assembly 116, after moving incoming product(s) to a grader, such as with the product movement assembly 114. The grading assembly 116 may be controlled with a grader controller, such as grader controller 118, which may receive instructions from one or more of the containment assembly controller 122 and the collected product optimization computing device 126.

The physical attribute data of the product(s) may include one or more of type, size, shape, mass, volume, weight, color, etc. In that regard, one or more sensor assemblies may be associated with the grading assembly 116 of the grader 106 for obtaining the physical attribute data. The sensor assemblies may obtain sensor data pertaining to physical attribute(s) of the collected product(s) and send such data to a collected product(s) data storage device writer, such as collected product(s) data storage device writer 236.

The method 702 may proceed to block 708, where physical attribute data of the collected product(s) is written to a data storage device corresponding to a collected product(s) transport component of a collected product(s). For instance, the physical attribute data of the collected product(s) may be written to a collected product(s) data storage device 234 corresponding to a collected product(s) transport component 232 of a collected product(s). The collected product(s) transport component 232 may be a flight of a conveyor (e.g., flight 332/flight 432) or another suitable component that travels with the collected product(s) (e.g., a bin, a robot arm, etc.).

A collected product(s) data storage device writer, such as collected product(s) data storage device writer 236, may be used to write the physical attribute data of the collected product(s) to the collected product(s) data storage device 234. The method may first include (optionally reading the collected product(s) data storage device 234 and) erasing any existing data on the collected product(s) data storage device 234 before writing the physical attribute data of the collected product(s) to the collected product(s) data storage device 234. In the examples shown in FIGS. 3, 4, and 5, the collected product(s) data storage device 234 is an RFID tag, and the collected product(s) data storage device writer 236 is an RFID tag (reader/)writer. However, other suitable storage devices or chips and corresponding data (reader/)writers may be used.

The method 702 may proceed to block 710, where the collected product(s) are transported with the collected product(s) transport component 232 to a containment machine, such as collected product(s) containment machine 110. The collected product(s) may be transported with a graded product movement system, such as graded product movement system 108. The graded product movement system 108 may be controlled via a conveyor controller, which may receive instructions from one or more of the grader controller 118, the containment assembly controller 122, and the collected product optimization computing device 126.

The method 702 may proceed to block 712, where a collected product(s) data storage device reader, such as the collected product(s) data storage device reader 238, reads physical attribute data from the collected product(s) data storage devices 234 corresponding to a collected product(s). As discussed above, the collected product(s) data storage device reader 238 may be positioned near an inlet of the collected product(s) containment machine 110 to retrieve data pertaining to the collected product(s) to be delivered into the containment machine. For instance, in the example shown in FIG. 4, data reader/writer device 438 is positioned near an inlet of the containment machine 410 to retrieve data pertaining to the collected product(s) to be delivered into the containment machine 410. Specifically, the data reader/writer device 438 is positioned above the containment hopper 446 to obtain data for each collected product(s) that is deposited into the hopper from the graded product movement system 408. In that regard, the containment machine 410 can obtain and use physical attribute data relevant to the deposited collected product(s) for managing containment of that collected product(s). Data pertaining to the collected product(s) delivered into the containment machine 410 can be used by the containment controller (e.g., see containment assembly controller 122) to optimize containment, e.g., to control, monitor, and/or manage aspects of the containment.

In that regard, the method 702 may proceed to block 714, which includes optimizing containment of collected product(s) using the corresponding physical attribute data. For instance, the physical attribute information of the collected product(s), retrieved from the collected product(s) data storage device 234 by the collected product(s) data storage device reader 238, may be used to code, label, or otherwise identify the packaged collected product(s). For instance, the containment assembly controller 122 may process the retrieved physical attribute information of the collected product(s) and instruct a labeler to print at least some of that information on a package label. In that regard, physical attribute data can be tied to the packaged, collected product(s) without the need for complicated transfer of information.

Specifically, no transfer of data between the grader 106 and the collected product(s) containment machine 110 is needed to associate physical attribute data of collected product(s) with a packaged collected product(s). Rather, the collected product management systems and methods disclosed herein provide a means to exchange product physical attribute information without the actual transference of data via a control or computing system. The physical attribute data may be written to a tag attached to the conveyor belt or other transport component, and data buffer or register manipulation in the packager/grader control system is not required. In that regard, power failures, missed bins, etc., do not adversely impact collected product(s) data retention. Once the data is written to the collected product(s) data storage device 234, the data stays with the storage device until it is erased.

The collected product(s) containment machine 110 can miss bags, skip bags, miss collection buckets, skip collection buckets, etc., or otherwise come out of sync with collected product(s) delivery. Yet using the systems and methods disclosed herein, data regarding the collected product(s) is collected just before or as the collected product(s) is deposited into the collected product(s) containment machine 110. Thus, the physical attribute information can be directly associated with the collected product(s) and used to code/label the packaged collected product(s) (e.g., the bag, bin, collection point, etc.). Accordingly, there is a 1:1 correlation between physical attribute data retrieved and the packaged collected product(s). The systems and methods disclosed herein provide a flexible method for data management while at the same time requiring a minimal amount of operator recognition or involvement.

Nevertheless, the collected product(s) data storage device reader 238 may relay such physical attribute information to another computing device, such as the collected product optimization computing device 126, the model management computing device 128, and/or the monitoring system 130. The collected product optimization computing device 126 (e.g., the containment optimization engine 612) may use the physical attribute information to determine any collected product(s) containment management recommendations or instructions, such as for optimizing one or more of grading, containment, labeling, and end use of the collected product(s).

For instance, the containment optimization engine 612 of the collected product optimization computing device 126 may output instructions to the containment assembly controller 122 and/or the grader controller 118 of the collected product(s) containment machine 110 and/or grader 106, respectively, for automatically or semi-automatically carrying out an action according to the instructions. The containment optimization engine 612 may also or instead output instructions to the containment optimization engine 612 to display a recommendation associated with the collected product(s) containment machine 110 and/or the grader 106 such that an operator may carry out any necessary actions.

Physical attribute data for a collected product(s) may also or instead be sent to or retrieved by the model management computing device 128 for training one or more containment optimization machine learning model(s) to output containment management recommendations or instructions. As discussed above, containment optimization machine learning model(s) may be trained to output a collected product(s) containment optimization plan that includes recommendations or instructions to adjust component(s) or setting(s) of the collected product(s) containment machine 110 and/or the grader 106 based on at least one of physical attribute data of collected product(s), containment machine settings, containment material data, grader settings, incoming product supply data, and product demand data.

As noted above, the physical attribute data for a collected product(s) may also be sent to the monitoring system 130. The monitoring system 130 may be generally configured to curate and provide information regarding the collected product(s) management system 102. For instance, the monitoring system 130 may use the physical attribute information to perform grader, conveyor, and/or containment machine analytics. As a specific example, physical attribute data for collected product(s) may be used to determine a yield to target analysis of packaged collected product(s), such as a percentage of packaged collected product(s) that are within a certain range or threshold of a physical attribute value (e.g., a packaged chicken breast, wing, thigh, and leg that are within a certain weight range).

FIG. 8 is a block diagram that illustrates aspects of an exemplary computing device 800 appropriate for use as a computing device of the present disclosure. While multiple different types of computing devices were discussed above, the exemplary computing device 800 describes various elements that are common to many different types of computing devices. While FIG. 8 is described with reference to a computing device that is implemented as a device on a network, the description below is applicable to servers, personal computers, mobile phones, smart phones, tablet computers, embedded computing devices, and other devices that may be used to implement portions of embodiments of the present disclosure. Some embodiments of a computing device may be implemented in or may include an application-specific integrated circuit (ASIC), a field-programmable gate array (FPGA), or other customized device. Moreover, those of ordinary skill in the art and others will recognize that the computing device 800 may be any one of any number of currently available or yet to be developed devices.

In its most basic configuration, the computing device 800 includes at least one processor 802 and a system memory 810 connected by a communication bus 808. Depending on the exact configuration and type of device, the system memory 810 may be volatile or nonvolatile memory, such as read only memory (“ROM”), random access memory (“RAM”), EEPROM, flash memory, or similar memory technology. Those of ordinary skill in the art and others will recognize that system memory 810 typically stores data and/or program modules that are immediately accessible to and/or currently being operated on by the processor 802. In this regard, the processor 802 may serve as a computational center of the computing device 800 by supporting the execution of instructions.

As further illustrated in FIG. 8, the computing device 800 may include a network interface 806 comprising one or more components for communicating with other devices over a network. Embodiments of the present disclosure may access basic services that utilize the network interface 806 to perform communications using common network protocols. The network interface 806 may also include a wireless network interface configured to communicate via one or more wireless communication protocols, such as Wi-Fi, 2G, 3G, LTE, WiMAX, Bluetooth, Bluetooth low energy, and/or the like. As will be appreciated by one of ordinary skill in the art, the network interface 806 illustrated in FIG. 8 may represent one or more wireless interfaces or physical communication interfaces described and illustrated above with respect to particular components of the computing device 800.

In the exemplary embodiment depicted in FIG. 8, the computing device 800 also includes a storage medium 804. However, services may be accessed using a computing device that does not include means for persisting data to a local storage medium. Therefore, the storage medium 804 depicted in FIG. 8 is represented with a dashed line to indicate that the storage medium 804 is optional. In any event, the storage medium 804 may be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

Suitable implementations of computing devices that include a processor 802, system memory 810, communication bus 808, storage medium 804, and network interface 806 are known and commercially available. For ease of illustration and because it is not important for an understanding of the claimed subject matter, FIG. 8 does not show some of the typical components of many computing devices. In this regard, the computing device 800 may include input devices, such as a keyboard, keypad, mouse, microphone, touch input device, touch screen, tablet, and/or the like. Such input devices may be coupled to the computing device 800 by wired or wireless connections including RF, infrared, serial, parallel, Bluetooth, Bluetooth low energy, USB, or other suitable connections protocols using wireless or physical connections. Similarly, the computing device 800 may also include output devices such as a display, speakers, printer, etc. Since these devices are well known in the art, they are not illustrated or described further herein.

While the concepts of the present disclosure are susceptible to various modifications and alternative forms, specific examples thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one example,” “an example,” “an exemplary example,” etc., indicate that the example described may include a particular feature, structure, or characteristic, but every example may or may not necessarily include that particular feature, structure, or characteristic. Moreover, such phrases are not necessarily referring to the same example. Further, when a particular feature, structure, or characteristic is described in connection with an example, it is submitted that it is within the knowledge of one skilled in the art to affect such feature, structure, or characteristic in connection with other examples whether or not explicitly described. Additionally, it should be appreciated that items included in a list in the form of “at least one A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Language such as “upstream”, “downstream”, “left”, “right”, “first”, “second”, etc., in the present disclosure is meant to provide orientation for the reader with reference to the drawings and is not intended to be the required orientation of the components or graphical images or to impart orientation limitations into the claims.

In the drawings, some structural or method features may be shown in specific arrangements and/or orderings. However, it should be appreciated that such specific arrangements and/or orderings may not be required. Rather, in some examples, such features may be arranged in a different manner and/or order than shown in the illustrative FIG. Additionally, the inclusion of a structural or method feature in a particular FIG. is not meant to imply that such feature is required in all examples and, in some examples, it may not be included or may be combined with other features.

Methods according to the above-described examples can be implemented using computer-executable instructions that are stored or otherwise available from computer-readable media of a computing device. Such instructions can comprise, for example, instructions and data which cause or otherwise configure a general purpose computer, special purpose computer, or special purpose processing device to perform a certain function or group of functions. Portions of computer resources used can be accessible over a network. The executable computer instructions may be, for example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, USB devices provided with non-volatile memory, networked storage devices, and so on.

Systems implementing methods according to this disclosure can comprise hardware, firmware and/or software, and can take any of a variety of form factors. Typical examples of such form factors include servers, laptops, smartphones, small form factor personal computers, personal digital assistants, and so on. The functionality described herein also can be embodied in peripherals or add-in cards. Such functionality can also be implemented on a circuit board among different chips or different processes executing in a single device, by way of further example. The instructions, media for conveying such instructions, computing resources for executing them, and other structures for supporting such computing resources are means for providing the functions described in these disclosures.

Various examples of the disclosure are discussed in detail above. While specific implementations are discussed, it should be understood that this description is for illustration purposes only. A person skilled in the relevant art will recognize that other components and configurations may be used without parting from the spirit and scope of the disclosure. Thus, the description and drawings herein are illustrative and are not to be construed as limiting. Numerous specific details are described to provide a thorough understanding of the disclosure. However, in certain instances, well-known or conventional details are not described in order to avoid obscuring the description. References to one or an example in the present disclosure can be references to the same example or any example; and, such references mean at least one of the examples.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various examples given in this specification.

Without intent to limit the scope of the disclosure, examples of machines, components, methods and their related results according to the examples of the present disclosure are given above. Note that titles or subtitles may be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions, will control.

The present disclosure may also reference quantities and numbers. Unless specifically stated, such quantities and numbers are not to be considered restrictive, but exemplary of the possible quantities or numbers associated with the present disclosure. Also in this regard, the present disclosure may use the term “plurality” to reference a quantity or number. In this regard, the term “plurality” is meant to be any number that is more than one, for example, two, three, four, five, etc. As used herein, the terms “about”, “approximately,” etc., in reference to a number, is used herein to include numbers that fall within a range of 10%, 5%, or 1% in either direction (greater than or less than) the number unless otherwise stated or otherwise evident from the context (except where such number would exceed 100% of a possible value).

While illustrative embodiments have been illustrated and described, it will be appreciated that various changes can be made therein without departing from the spirit and scope of the invention.

The embodiments of the invention in which an exclusive property or privilege is claimed are defined as follows:

1. A collected product(s) management system for managing containment of collected product(s), comprising:
a grader configured to associate a collected product(s) having predetermined collected product(s) physical attribute information with a collected product(s) transport component;
a collected product(s) physical attribute information system configured to write collected product(s) physical attribute information regarding a collected product(s) to a data storage device and obtain the collected product(s) physical attribute information from the data storage device, each data storage device associated with a collected product(s) transport component; and
a product containment machine configured to contain collected product(s) associated with a collected product(s) transport component and identify the contained collected product(s) with the collected product(s) physical attribute information obtained from the data storage device of the associated collected product(s) transport component.

2. The collected product(s) management system of claim 1, wherein the collected product(s) physical attribute information system includes a sensor assembly configured to obtain sensor data regarding the collected product(s) physical attribute information.

3. The collected product(s) management system of claim 1, wherein the collected product(s) physical attribute information system comprises an RFID reader and an RFID tag secured within the collected product(s) transport component, the RFID reader positioned relative to an inlet of the product containment machine to read the RFID tag.

4. The collected product(s) management system of claim 1, wherein a first data storage device is associated with a first collected product(s) transport component configured to maintain separation of a first collected product(s) from a second collected product(s).

5. The collected product(s) management system of claim 1, wherein the collected product(s) transport component is a flight of an endless conveyor belt.

6. The collected product(s) management system of claim 1, wherein the product containment machine includes a labeler configured to add collected product(s) physical attribute information to a containment device of each corresponding collected product(s).

7. The collected product(s) management system of claim 1, wherein the collected product(s) are food products, and wherein the collected product(s) physical attribute information is at least one of type, size, weight, volume, count, and color.

8. The collected product(s) management system of claim 1, wherein the grader comprises:
a grader assembly having a sensor assembly configured to obtain sensor data regarding collected product(s) physical attribute information and a product sorting assembly configured to move at least one product of an incoming supply of products to a first designated collected product(s) location to define a first collected product(s); and
a grader controller configured to activate the product sorting assembly to move the at least one product of the incoming supply of products to the first designated collected product(s) location to define the first collected product(s) based on collected product(s) physical attribute information of the first collected product(s).

9. The collected product(s) management system of claim 8, wherein the first designated collected product(s) location is associated with a first collected product(s) transport component of a product movement assembly, the first collected product(s) transport component configured to segregate the first collected product(s) from a second collected product(s) during movement of the first and second collected product(s) from the grader to the product containment machine.

10. The collected product(s) management system of claim 9, wherein a first data storage device is associated with the first collected product(s) transport component to store data regarding collected product(s) physical attribute information of the first collected product(s).

11. The collected product(s) management system of claim 1, further comprising:
a processor; and
a memory storing instructions that, when executed by the processor, cause a computing device of the collected product management system to:
process collected product(s) physical attribute information; and
output a collected product(s) containment optimization plan including at least one of recommendations and instructions for adjusting at least one of the containment machine and the grader based on at least one of collected product(s) physical attribute information, containment machine settings, containment machine material data, grader settings, incoming product supply data, and product demand data.

12. The collected product management system of claim 11, wherein the collected product(s) containment optimization plan includes at least one of recommendations and instructions for the grader to use a certain grading specification for collected product(s).

13. The collected product management system of claim 11, wherein the collected product(s) containment optimization plan includes at least one of recommendations and instructions to sort packaged, labeled collected product(s) of a first classification to a first end use and packaged, labeled collected product(s) of a second classification to a second end use.

14. The collected product management system of claim 11, wherein the collected product(s) containment optimization plan includes at least one of recommendations and instructions to adjust a product containment size.

15. The collected product management system of claim 11, wherein the memory storing instructions that, when executed by the processor, further cause a computing device of the collected product(s) management system to:
determine a data storage read count for a period; and
output instructions to a controller of the product containment machine to at least one of adjust a speed of the product containment and stop the product containment machine if the data storage read count is less than an expected count for the period.

16. The collected product management system of claim 11, wherein the memory storing instructions that, when executed by the processor, further cause a computing device of the collected product(s) management system to:
execute one or more containment optimization machine learning models to output the collected product(s) containment optimization plan using at least one of collected product(s) physical attribute information, containment machine settings, containment machine material data, grader settings, incoming product supply data, and product demand data as input.

17. A collected product(s) management system for managing containment of a collected product(s), comprising:
a grader configured to associate a collected product(s) with collected product(s) physical attribute information from an incoming supply of products;
a collected product(s) physical attribute information system configured to write collected product(s) physical attribute information associated with a collected product(s) from the incoming supply of products to a data storage device and obtain the collected product(s) physical attribute information from the data storage device;
a product containment machine configured to contain each collected product(s) associated with collected product(s) physical attribute information and identify each contained collected product(s) with corresponding collected product(s) physical attribute information obtained from the corresponding data storage device; and
a product movement assembly configured to transport each collected product(s) from the grader to the product containment machine while associating each collected product(s) with the corresponding data storage device.

18. The collected product management system of claim 17, wherein a first data storage device is associated with a first collected product(s) transport component of the product movement assembly, the first collected product(s) transport component configured to maintain separation of a first collected product(s) from a second collected product(s).

19. The collected product management system of claim 18, wherein the first collected product(s) transport component is a flight of an endless conveyor belt.

20. The collected product management system of claim 17, wherein the collected product(s) physical attribute information system includes a sensor assembly configured to obtain sensor data regarding the collected product(s) physical attribute information.
